(19) United States
(12) Patent Application Publication
(10) Pub. No.: US 2025/0051318 A1
(43) Pub. Date: Feb. 13, 2025

(54) Processes For The Preparation Of Oxadiazolylamino Benzodiazepine Derivatives

(71) Applicant: Enanta Pharmaceuticals, Inc., Watertown, MA (US)

(72) Inventors: Scott CRAWFORD, Southborough, MA (US); Weipeng HU, Brighton, MA (US); Joseph HELBLE, Hudson, MA (US); Cody SHEPPARD, Wilmington, MA (US); Tao WANG, Berkeley Heights, NJ (US)

(21) Appl. No.: 18/797,587

(22) Filed: Aug. 8, 2024

Related U.S. Application Data
(60) Provisional application No. 63/531,701, filed on Aug. 9, 2023.

(51) Int. Cl. C07D 413/12

(52) U.S. Cl. CPC C07D 413/12

(57) ABSTRACT

The present invention relates to processes for preparing Compound (I):

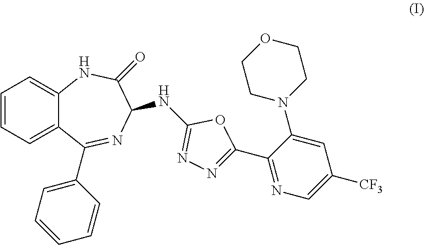 [SMILES: O=C1Nc2ccccc2C(c2ccccc2)=N[C@@H]1Nc1nnc(-c2ncc(C(F)(F)F)cc2N2CCOCC2)o1]

or a pharmaceutically acceptable salt or solvate thereof. Compound (I) and pharmaceutical compositions are useful as Respiratory Syncytial Virus (RSV) inhibitors.

### RELATED APPLICATION

This application claims the benefit of U.S. Provisional Application No. 63/531,701, filed Aug. 9, 2023. The entire teachings of the above application are incorporated herein by reference.

### TECHNICAL FIELD

The present invention relates to processes in the preparation of biologically active compounds and pharmaceutical compositions useful as Respiratory Syncytial Virus (RSV) inhibitors. Specifically, the present invention relates to processes to prepare oxadiazolylamino benzodiazepine derivatives that can inhibit RSV activities and for treating RSV infection.

### BACKGROUND OF THE INVENTION

Human respiratory syncytial virus (HRSV) is a negative-sense, single stranded, RNA paramyxovirus (KM. Empey, et al, Rev. Anti-Infective Agents, 2010, 50 (1 May), 1258-1267). RSV is the leading cause of acute lower respiratory tract infections (ALRI) and affects patients of all ages. The symptoms in adults are usually not severe and are typically analogous to a mild cold. However, in infants and toddlers the virus can cause lower respiratory tract infections including bronchiolitis or pneumonia with many of them requiring hospitalization. Nearly all children have been infected by age 3. There are known high-risk groups that infection with RSV is more likely to progress into the ALRL Premature infants and/or infants suffering from lung or cardiac disease are at the highest risk to develop ALRL Additional high-risk groups include the elderly, adults with chronic heart and/or lung disease, stem cell transplant patients and the immunosuppressed.

Palivizumab is a monoclonal antibody that is used prophylactically to prevent HRSV infection in high-risk infants, e.g. premature infants, and infants with cardiac and/or lung disease. The high cost of palivizumab treatment limits its use for general purposes. Ribavirin has also been used to treat HRSV infections, but its effectiveness is limited. There is a major medical need for new and effective HRSV treatments that can be used generally by all population types and ages.

There have been several RSV fusion inhibitors that have been disclosed in the following publications: WO2010/103306, WO2012/068622, WO2013/096681, WO2014/060411, WO2013/186995, WO2013/186334, WO2013/186332, WO2012/080451, WO2012/080450, WO2012/080449, WO2012/080447, WO2012/080446, WO 2015/110446, WO 2017/009316, WO 2017/015449, and J Med. Chem. 2015, 58, 1630-1643. Examples of other N-protein inhibitors for treatment of HRSV have been disclosed in the following publications: WO2004/026843, J Med. Chem. 2006, 49, 2311-2319, and J. Med. Chem. 2007, 50, 1685-1692. Examples of L-protein inhibitors for HRSV have been disclosed in the following publications: WO2011/005842, WO2005/042530, Antiviral Res. 2005, 65, 125-131, and Bioorg. Med. Chem. Lett. 2013, 23, 6789-6793. Examples of nucleosides/polymerase inhibitors have been disclosed in the following publications: WO2013/242525 and J Med. Chem. 2015, 58, 1862-1878.

There is a need for the development of therapeutic agents for treating HRSV and methods of producing such agents.

### SUMMARY OF THE INVENTION

In a principal embodiment, the invention provides a method for preparing Compound (I):

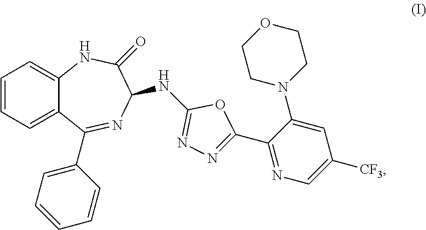 [SMILES: O=C1Nc2ccccc2C(c2ccccc2)=N[C@@H]1Nc1nnc(-c2ncc(C(F)(F)F)cc2N2CCOCC2)o1]

which is a potent inhibitor of HRSV.

The methods of the invention provide Compound (I) in increased product yield and purity compared to methods disclosed in WO 2018/152413.

### DETAILED DESCRIPTION OF THE INVENTION

The present invention provides methods for preparing Compound (1):

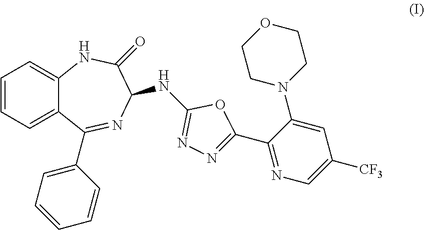 [SMILES: O=C1Nc2ccccc2C(c2ccccc2)=N[C@@H]1Nc1nnc(-c2ncc(C(F)(F)F)cc2N2CCOCC2)o1]

In one embodiment, the invention provides a process for preparing Compound (I),

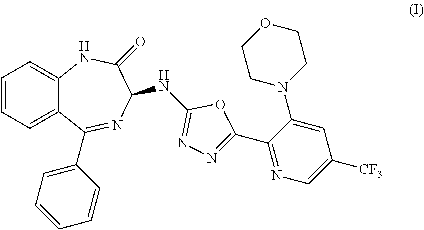 [SMILES: O=C1Nc2ccccc2C(c2ccccc2)=N[C@@H]1Nc1nnc(-c2ncc(C(F)(F)F)cc2N2CCOCC2)o1]

comprising the steps of:
(a) reacting compound (A) with 1,1′-carbonyldiimidazole, to produce compound (B):

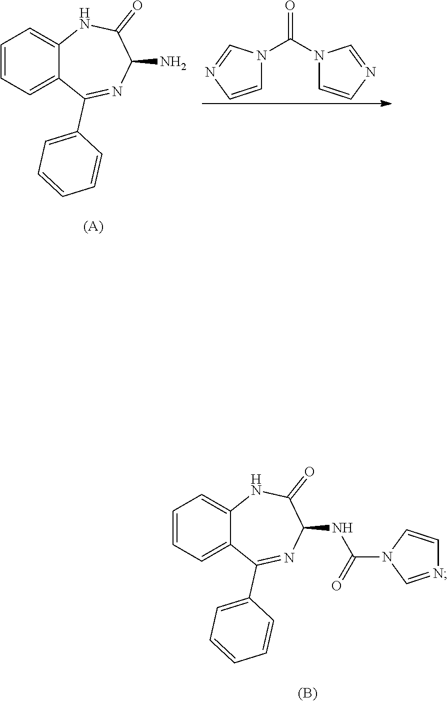

(b) reacting compound (B) with compound (W), to produce compound (C):

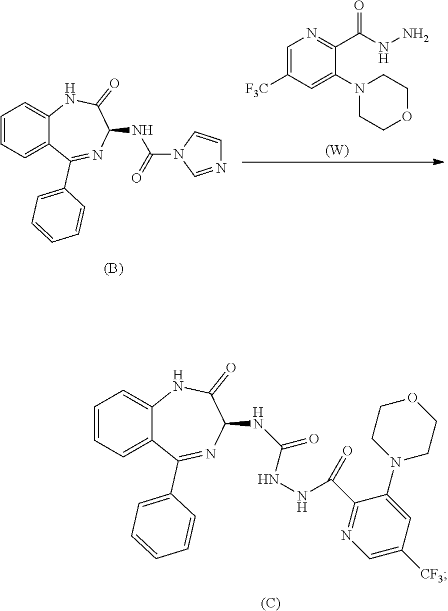 [SMILES: O=C1Nc2ccccc2C(c2ccccc2)=N[C@@H]1NC(=O)n1ccnc1 ; NNC(=O)c1ncc(C(F)(F)F)cc1N1CCOCC1 ; "(W)" ; "(B)" ; O=C(NNC(=O)c1ncc(C(F)(F)F)cc1N1CCOCC1)N[C@H]1N=C(c2ccccc2)c2ccccc2NC1=O ; "(C)"]

and
(c) reacting compound (C) with a dehydration agent, to produce Compound (I):

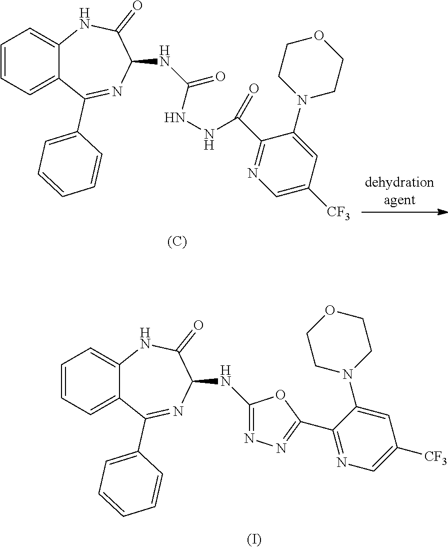 [SMILES: O=C(NNC(=O)c1ncc(C(F)(F)F)cc1N1CCOCC1)N[C@H]1N=C(c2ccccc2)c2ccccc2NC1=O ; "(C)" ; O=C1Nc2ccccc2C(c2ccccc2)=N[C@@H]1Nc1nnc(-c2ncc(C(F)(F)F)cc2N2CCOCC2)o1 ; "(I)"]

### Step (a)

In preferred embodiments, step (a) is conducted in a suitable solvent, such as, but not limited to, acetonitrile, dichloromethane, dimethylformamide, dimethyl sulfoxide, tetrahydrofuran, dimethylacetamide, N-methyl-2-pyrrolidone, sulfolane, dioxane or a mixture of two or more thereof. Preferably the solvent is acetonitrile.

In preferred embodiments, step (a) is conducted at a temperature from about 20° C. to about 80° C., preferably from about 35° C. to about 45° C., and more preferably about 40° C. In certain embodiments, step (a) takes place for a period from about one hour to about 24 hours, preferably about 3 hours to about 6 hours, and more preferably about 4 hours.

In preferred embodiments, the process of the invention further comprises isolating compound (B), preferably in a substantially pure form.

### Step (b)

In preferred embodiments, step (b) is conducted in a suitable solvent, such as, but not limited to, acetonitrile, dimethylformamide, dimethylsulfoxide, tetrahydrofuran, N-methyl-2-pyrrolidone, sulfolane, dioxane or a mixture of two or more thereof. Preferably the solvent is N-methyl-2-pyrrolidone.

In preferred embodiments, step (b) is conducted at a temperature from about 10° C. to about 50° C., preferably from about 30° C. to about 50° C., and more preferably about 35° C. In one embodiment, step (a) takes place for a period from about 6 hours to about 72 hours, preferably about 24 hours to about 48 hours.

In a preferred embodiment, the process of the invention further comprises isolating compound (C), preferably in a substantially pure form.

In certain embodiments, the invention further provides a method of purifying compound (C) by precipitation. The method includes dissolving compound (C) in a suitable solvent at a temperature from about 20° C. to about 30° C., preferably about 25° C. An anti-solvent is then charged to the resulting solution, thereby inducing precipitation of compound (C). Suitable solvents for dissolution include, but are not limited to, acetonitrile, dichloromethane, dimethyl sulfoxide, N-methyl-2-pyrrolidone and ethanol. Preferably the solvent is ethanol. Suitable anti-solvents for precipitation include, but are not limited to, water, heptane, and methyl t-butyl ether. Preferably the anti-solvent is heptane.

### Step (c)

In a preferred embodiment, step (c) is conducted in a suitable solvent, such as, but not limited to, acetonitrile, dichloromethane, chloroform, dimethylformamide, dimethylsulfoxide, dioxane, ethyl acetate, heptane, hexane, methyl t-butyl ether, tetrahydrofuran, 2-methyltetrahydrofuran, or toluene, or a mixture of two or more thereof. Preferably the solvent is tetrahydrofuran.

In preferred embodiments, step (c) is conducted in the presence of a suitable base, such as, but not limited to, 1,4-diazabicyclo[2.2.2]octane (DABCO), triethylamine, 1,8-diazabicyclo[5.4.0]undec-7-ene (DBU), 1-methylimidazole, diisopropylethylamine, sodium bicarbonate, potassium phosphate, sodium hydroxide or N-methylmorpholine. A preferred base is 1,4-diazabicyclo[2.2.2]octane (DABCO).

In preferred embodiments, step (c) is conducted at a temperature from about −10° C. to about 60° C., preferably from about 10° C. to about 40° C., and more preferably about 25° C. In one embodiment, step (c) takes place for a period from about 1 hour to about 24 hours, preferably about 3 hours to about 8 hours, and more preferably about 4 hours.

Suitable dehydration agents include, but are not limited to N,N-dimethylsulfamoyl chloride, p-toluenesulfonyl chloride, methyl N-(triethylammoniumsulfonyl) carbamate (Burgess reagent), phosphorus trichloride, phosphorus pentachloride, and phosphorus oxychloride. A preferred dehydration agent is N,N-dimethylsulfamoyl chloride.

In preferred embodiments, the process of the invention further comprises isolating Compound (I), preferably in a substantially pure form.

In certain embodiments, the invention further provides a method of purifying Compound (I) by recrystallization. The method includes dissolving Compound (I) in a first solvent at a temperature from about 20° C. to about 60° C., preferably from about 25° C. to about 40° C., and more preferably about 30° C. The first solvent is then replaced with a second solvent, which has lower solubility for the desired form, thereby inducing recrystallization of Compound (I). Suitable first solvents for dissolving Compound (I) prior to recrystallization include ethyl acetate, ethanol, methanol, 2-methyltetrahydrofuran, dimethyl sulfoxide, dimethylformamide, and mixtures of two or more thereof. Preferably the first solvent is acetone, ethanol or a mixture thereof. Suitable second solvents for inducing crystallization include toluene, heptane, ethanol, isopropanol, methyl t-butyl ether, water and mixtures of two or more thereof. Preferably the second solvent is ethanol, heptane, methyl t-butyl ether or a combination thereof. It is to be understood that when the first solvent is ethanol, the second solvent is not ethanol. Compound (I) is preferably isolated as an anhydrous form.

### Definitions

Listed below are definitions of various terms used to describe this invention. These definitions apply to the terms as they are used throughout this specification and claims, unless otherwise limited in specific instances, either individually or as part of a larger group.

The term “aryl,” as used herein, refers to a mono- or polycyclic carbocyclic ring system comprising at least one aromatic ring. Preferred aryl groups are C₆-C₁₂-aryl groups, including, but not limited to, phenyl, naphthyl, tetrahydronaphthyl, indanyl, and indenyl. A polycyclic aryl is a polycyclic ring system that comprises at least one aromatic ring. Polycyclic aryls can comprise fused rings, covalently attached rings or a combination thereof.

The term “heteroaryl,” as used herein, refers to a mono- or polycyclic aromatic radical having one or more ring atom selected from S, O and N; and the remaining ring atoms are carbon, wherein any N or S contained within the ring may be optionally oxidized. In certain embodiments, a heteroaryl group is a 5- to 10-membered heteroaryl, such as a 5- or 6-membered monocyclic heteroaryl or an 8- to 10-membered bicyclic heteroaryl. Heteroaryl groups include, but are not limited to, pyridinyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, benzimidazolyl, benzoxazolyl, quinoxalinyl. A polycyclic heteroaryl can comprise fused rings, covalently attached rings or a combination thereof. A heteroaryl group can be C-attached or N-attached where possible.

In accordance with the invention, aryl and heteroaryl groups can be substituted or unsubstituted.

The term “bicyclic aryl” or “bicyclic heteroaryl” refers to a ring system consisting of two rings wherein at least one ring is aromatic; and the two rings can be fused or covalently attached.

The term “alkyl” as used herein, refers to saturated, straight- or branched-chain hydrocarbon radicals. “C₁-C₄ alkyl,” “C₁-C₆ alkyl,” “C₁-C₈ alkyl,” “C₁-C₁₂ alkyl,” “C₂-C₄ alkyl,” and “C₃-C₆ alkyl,” refer to alkyl groups containing from 1 to 4, 1 to 6, 1 to 8, 1 to 12, 2 to 4 and 3 to 6 carbon atoms respectively. Examples of alkyl groups include, but are not limited to, methyl, ethyl, propyl, isopropyl, n-butyl, sec-butyl, tert-butyl, n-pentyl, neopentyl, n-hexyl, n-heptyl and n-octyl radicals.

The term “alkenyl” as used herein, refers to straight- or branched-chain hydrocarbon radicals having at least one carbon-carbon double bond. “C₂-C₈ alkenyl,” “C₂-C₁₂ alkenyl,” “C₂-C₄ alkenyl,” “C₃-C₄ alkenyl,” and “C₃-C₆ alkenyl,” refer to alkenyl groups containing from 2 to 8, 2 to 12, 2 to 4, 3 to 4 or 3 to 6 carbon atoms respectively. Alkenyl groups include, but are not limited to, ethenyl, propenyl, butenyl, 2-methyl-2-buten-2-yl, heptenyl, octenyl, and the like.

The term “alkynyl” as used herein, refers to straight- or branched-chain hydrocarbon radicals having at least one carbon-carbon triple bond. “C₂-C₈ alkynyl,” “C₂-C₁₂ alkynyl,” “C₂-C₄ alkynyl,” “C₃-C₄ alkynyl,” and “C₃-C₆ alkynyl,” refer to alkynyl groups containing from 2 to 8t, 2 to 12, 2 to 4, 3 to 4 or 3 to 6 carbon atoms respectively. Representative alkynyl groups include, but are not limited to, ethynyl, 2-propynyl, 2-butynyl, heptynyl, octynyl, and the like.

The term “cycloalkyl”, as used herein, refers to a monocyclic or polycyclic saturated carbocyclic ring, such as a bi- or tri-cyclic fused, bridged or spiro system. The ring carbon atoms are optionally oxo-substituted or optionally substituted with an exocyclic olefinic double bond. Preferred cycloalkyl groups include C₃-C₁₂ cycloalkyl, C₃-C₆ cycloalkyl, C₃-C₈ cycloalkyl and C₄-C₇ cycloalkyl. Examples of cycloalkyl include, but are not limited to, cyclopropyl, cyclobutyl, cyclopentyl, cyclohexyl, cyclopentyl, cyclooctyl, 4-methylene-cyclohexyl, bicyclo[2.2.1]heptyl, bicyclo[3.1.0]hexyl, spiro[2.5]octyl, 3-methylenebicyclo[3.2.1]octyl, spiro[4.4]nonanyl, and the like.

The term “cycloalkenyl”, as used herein, refers to monocyclic or polycyclic carbocyclic ring, such as a bi- or tri-cyclic fused, bridged or spiro system having at least one carbon-carbon double bond. The ring carbon atoms are optionally oxo-substituted or optionally substituted with an exocyclic olefinic double bond. Preferred cycloalkenyl groups include C₃-C₁₂ cycloalkenyl, C₄-C₁₂-cycloalkenyl, C₃-C₈ cycloalkenyl, C₄-C₈ cycloalkenyl and C₅-C₇ cycloalkenyl groups. Examples of cycloalkenyl include, but are not limited to, cyclopropenyl, cyclobutenyl, cyclopentenyl, cyclohexenyl, cycloheptenyl, cyclooctenyl, bicyclo[2.2.1]hept-2-enyl, bicyclo[3.1.0]hex-2-enyl, spiro[2.5]oct-4-enyl, spiro[4.4]non-2-enyl, bicyclo[4.2.1]non-3-en-12-yl, and the like.

As used herein, the term “arylalkyl” means a functional group wherein an alkylene chain is attached to an aryl group, e.g., —(CH₂)ₙ-phenyl, where n is 1 to 12, preferably 1 to 6 and more preferably 1 or 2. The term “substituted arylalkyl” means an arylalkyl functional group in which the aryl group is substituted. Similarly, the term “heteroarylalkyl” means a functional group wherein an alkylene chain, is attached to a heteroaryl group, e.g., —(CH₂) n-heteroaryl, where n is 1 to 12, preferably 1 to 6 and more preferably 1 or 2. The term “substituted heteroarylalkyl” means a heteroarylalkyl functional group in which the heteroaryl group is substituted.

As used herein, the term “alkoxy” refers to a radical in which an alkyl group having the designated number of carbon atoms is connected to the rest of the molecule via an oxygen atom. Alkoxy groups include C₁-C₁₂-alkoxy, C₁-C₈-alkoxy, C₁-C₆-alkoxy, C₁-C₄-alkoxy and C₁-C₃-alkoxy groups. Examples of alkoxy groups includes, but are not limited to, methoxy, ethoxy, n-propoxy, 2-propoxy (isopropoxy) and the higher homologs and isomers. Preferred alkoxy is C₁-C₃ alkoxy.

An “aliphatic” group is a non-aromatic moiety comprised of any combination of carbon atoms, hydrogen atoms, halogen atoms, oxygen, nitrogen or other atoms, and optionally contains one or more units of unsaturation, e.g., double and/or triple bonds. Examples of aliphatic groups are functional groups, such as alkyl, alkenyl, alkynyl, O, OH, NH, NH₂, C(O), S(O)₂, C(O)O, C(O)NH, OC(O)O, OC(O)NH, OC(O)NH₂, S(O)₂NH, S(O)₂NH₂, NHC(O)NH₂, NHC(O)C(O) NH, NHS(O)₂NH, NHS(O)₂NH₂, C(O)NHS(O)₂. C(O)NHS(O)₂NH or C(O)NHS(O)₂NH₂, and the like, groups comprising one or more functional groups, non-aromatic hydrocarbons (optionally substituted), and groups wherein one or more carbons of a non-aromatic hydrocarbon (optionally substituted) is replaced by a functional group. Carbon atoms of an aliphatic group can be optionally oxo-substituted. An aliphatic group may be straight chained, branched, cyclic, or a combination thereof and preferably contains between about 1 and about 24 carbon atoms, more typically between about 1 and about 12 carbon atoms. In addition to aliphatic hydrocarbon groups, as used herein, aliphatic groups expressly include, for example, alkoxyalkyls, polyalkoxyalkyls, such as polyalkylene glycols, polyamines, and polyimines, for example. Aliphatic groups may be optionally substituted.

The terms “heterocyclic” and “heterocycloalkyl” can be used interchangeably and refer to a non-aromatic ring or a polycyclic ring system, such as a bi- or tri-cyclic fused, bridged or spiro system, where (i) each ring system contains at least one heteroatom independently selected from oxygen, sulfur and nitrogen, (ii) each ring system can be saturated or unsaturated (iii) the nitrogen and sulfur heteroatoms may optionally be oxidized, (iv) the nitrogen heteroatom may optionally be quaternized, (v) any of the above rings may be fused to an aromatic ring, and (vi) the remaining ring atoms are carbon atoms which may be optionally oxo-substituted or optionally substituted with exocyclic olefinic double bond. Representative heterocycloalkyl groups include, but are not limited to, 1,3-dioxolane, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, quinoxalinyl, pyridazinonyl, 2-azabicyclo[2.2.1]-heptyl, 8-azabicyclo[3.2.1]octyl, 5-azaspiro[2.5]octyl, 2-oxa-7-azaspiro[4.4]nonanyl, 7-oxooxepan-4-yl, and tetrahydrofuryl. Such heterocyclic or heterocycloalkyl groups may be further substituted. A heterocycloalkyl or heterocyclic group can be C-attached or N-attached where possible.

It is understood that any alkyl, alkenyl, alkynyl, alicyclic, cycloalkyl, cycloalkenyl, aryl, heteroaryl, heterocyclic, aliphatic moiety or the like described herein can also be a divalent or multivalent group when used as a linkage to connect two or more groups or substituents, which can be at the same or different atom(s). One of skill in the art can readily determine the valence of any such group from the context in which it occurs.

The term “substituted” refers to substitution by independent replacement of one, two, or three or more of the hydrogen atoms with substituents including, but not limited to, —F, —Cl, —Br, —I, —OH, C₁-C₁₂-alkyl; C₂-C₁₂-alkenyl, C₂-C₁₂-alkynyl, —C₃-C₁₂-cycloalkyl, protected hydroxy, —NO₂, —N₃, —CN, —NH₂, protected amino, oxo, thioxo, —NH—C₁-C₁₂-alkyl, —NH—C₂-C₈-alkenyl, —NH—C₂-C₈-alkynyl, —NH—C₃-C₁₂-cycloalkyl, —NH-aryl,—NH-heteroaryl, —NH-heterocycloalkyl, -dialkylamino, -diarylamino, -diheteroarylamino, -O—C₁-C₁₂-alkyl, -O—C₂-C₈-alkenyl, -O—C₂-C₈-alkynyl, -O—C₃-C₁₂-cycloalkyl, -O-aryl, -O-heteroaryl, -O-heterocycloalkyl, —C(O)—C₁-C₁₂-alkyl, —C(O)—C₂-C₈-alkenyl, —C(O)—C₂-C₈-alkynyl, —C(O)—C₃-C₁₂-cycloalkyl, —C(O)-aryl, —C(O)-heteroaryl, -C(O)-heterocycloalkyl, —CONH₂, —CONH—C₁-C₁₂-alkyl, —CONH—C₂-C₈-alkenyl, —CONH—C₂-C₈-alkynyl, —CONH—C₃-C₁₂-cycloalkyl, —CONH-aryl, —CONH-heteroaryl, -CONH-heterocycloalkyl, -OCO₂-C₁-C₁₂-alkyl, -OCO₂-C₂-C₈-alkenyl, —OCO₂-C₂-C₈-alkynyl, -OCO₂-C₃-C₁₂-cycloalkyl, -OCO₂-aryl, -OCO₂-heteroaryl, -OCO₂-heterocycloalkyl, —CO₂-C₁-C₁₂ alkyl, —CO₂-C₂-C₈ alkenyl, —CO₂-C₂-C₈ alkynyl, —CO₂-C₃-C₁₂-cycloalkyl, —CO₂-aryl, —CO₂-heteroaryl, —CO₂-heterocyloalkyl, -OCONH₂, -OCONH—C₁-C₁₂-alkyl, -OCONH—C₂-C₈-alkenyl, -OCONH—C₂-C₈-alkynyl, -OCONH—C₃-C₁₂-cycloalkyl, -OCONH-aryl, -OCONH-heteroaryl, -OCONH-heterocycloalkyl, —NHC(O) H, —NHC(O)—C₁-C₁₂-alkyl, —NHC(O)—C₂-C₈-alkenyl, —NHC(O)—C₂-C₈-alkynyl, -NHC(O)—C₃-C₁₂-cycloalkyl, —NHC(O)-aryl, —NHC(O)-heteroaryl, —NHC(O)-heterocycloalkyl, -NHCO₂-C₁-C₁₂-alkyl, —NHCO₂-C₂-C₈-alkenyl, —NHCO₂-C₂-C₈-alkynyl, —NHCO₂-C₃-C₁₂-cycloalkyl, —NHCO₂-aryl, —NHCO₂-heteroaryl, —NHCO₂-heterocycloalkyl, —NHC(O)NH₂, —NHC(O)NH—C₁-C₁₂-alkyl, —NHC(O)NH—C₂-C₈-alkenyl, —NHC(O)NH—C₂-C₈-alkynyl, —NHC(O)NH—C₃-C₁₂-cycloalkyl, —NHC(O)NH-aryl, -NHC(O)NH-heteroaryl, —NHC(O)NH-heterocycloalkyl, —NHC(S)NH₂, —NHC(S)NH—C₁-C₁₂-alkyl, —NHC(S)NH—C₂-C₈-alkenyl, —NHC(S)NH—C₂-C₈-alkynyl, —NHC(S)NH—C₃-C₁₂-cycloalkyl, —NHC(S)NH-aryl, —NHC(S)NH-heteroaryl, —NHC(S)NH-heterocycloalkyl, -NHC(NH)NH₂, —NHC(NH)NH—C₁-C₁₂-alkyl, —NHC(NH)NH—C₂-C₈-alkenyl, —NHC(NH)NH—C₂-C₈-alkynyl, —NHC(NH)NH—C₃-C₁₂-cycloalkyl, —NHC(NH)NH-aryl, —NHC(NH)NH-heteroaryl, —NHC(NH)NH-heterocycloalkyl, -NHC(NH)—C₁-C₁₂-alkyl, —NHC(NH)—C₂-C₈-alkenyl, —NHC(NH)—C₂-C₈-alkynyl, —NHC(NH)—C₃-C₁₂-cycloalkyl, —NHC(NH)-aryl, —NHC(NH)-heteroaryl, —NHC(NH)-heterocycloalkyl, -C(NH) NH₂, —C(NH) NH—C₁-C₁₂-alkyl, —C(NH) NH—C₂-C₈-alkenyl, —C(NH) NH—C₂-C₈-alkynyl, —C(NH) NH—C₃-C₁₂-cycloalkyl, —C(NH) NH-aryl, —C(NH) NH-heteroaryl, —C(NH) NH-heterocycloalkyl, -S(O)—C₁-C₁₂-alkyl, -S(O)—C₂-C₈-alkenyl, -S(O)—C₂-C₈-alkynyl, -S(O)—C₃-C₁₂-cycloalkyl, -S(O)-aryl, -S(O)-heteroaryl, -S(O)-heterocycloalkyl, -SO₂NH₂, -SO₂NH—C₁-C₁₂-alkyl, -SO₂NH—C₂-C₈-alkenyl, -SO₂NH—C₂-C₈-alkynyl, -SO₂-C₁-C₁₂-alkyl, -SO₂-C₂-C₈-alkenyl, -SO₂-C₂-C₈-alkynyl, -SO₂-C₃-C₁₂-cycloalkyl, -SO₂-aryl, -SO₂-heteroaryl, -SO₂-heterocycloalkyl, -SO₂NH—C₃-C₁₂-cycloalkyl, -SO₂NH-aryl, -SO₂NH-heteroaryl, -SO₂NH-heterocycloalkyl, —NHSO₂-C₁-C₁₂-alkyl, —NHSO₂-C₂-C₈-alkenyl, —NHSO₂-C₂-C₈-alkynyl, —NHSO₂-C₃-C₁₂-cycloalkyl, —NHSO₂-aryl, —NHSO₂-heteroaryl, —NHSO₂-heterocycloalkyl, —CH₂NH₂, —CH₂SO₂CH₃, -aryl, -arylalkyl, -heteroaryl, -heteroarylalkyl, -heterocycloalkyl, -C₃-C₁₂-cycloalkyl, polyalkoxyalkyl, polyalkoxy, -methoxymethoxy, -methoxyethoxy, -SH, -S-C₁-C₁₂-alkyl, -S-C₂-C₈-alkenyl, -S-C₂-C₈-alkynyl, -S-C₃-C₁₂-cycloalkyl, -S-aryl, -S-heteroaryl, -S-heterocycloalkyl, or methylthio-methyl. In certain embodiments, the substituents are independently selected from halo, preferably Cl and F; C₁-C₄-alkyl, preferably methyl and ethyl; halo-C₁-C₄-alkyl, such as fluoromethyl, difluoromethyl, and trifluoromethyl; C₂-C₄-alkenyl; halo-C₂-C₄-alkenyl; C₃-C₆-cycloalkyl, such as cyclopropyl; C₁-C₄-alkoxy, such as methoxy and ethoxy; halo-C₁-C₄-alkoxy, such as fluoromethoxy, difluoromethoxy, and trifluoromethoxy; -CN; -OH; NH₂; C₁-C₄-alkylamino; di(C₁-C₄-alkyl)amino; and NO₂. It is understood that an aryl, heteroaryl, alkyl, alkenyl, alkynyl, cycloalkyl, or heterocycloalkyl in a substituent can be further substituted. In certain embodiments, a substituent in a substituted moiety is additionally optionally substituted with one or more groups, each group being independently selected from C₁-C₄-alkyl; -CF₃, -OCH₃, -OCF₃, —F, —Cl, —Br, -I, —OH, —NO₂, —CN, and —NH₂. Preferably, a substituted alkyl or alkoxy group is substituted with one or more halogen atoms, more preferably one or more fluorine or chlorine atoms.

The term “halo” or halogen” alone or as part of another substituent, as used herein, refers to a fluorine, chlorine, bromine, or iodine atom.

The term “optionally substituted”, as used herein, means that the referenced group may be substituted or unsubstituted. In one embodiment, the referenced group is optionally substituted with zero substituents, i.e., the referenced group is unsubstituted. In another embodiment, the referenced group is optionally substituted with one or more additional group(s) individually and independently selected from groups described herein.

The term “hydrogen” includes hydrogen and deuterium. In addition, the recitation of an element includes all isotopes of that element so long as the resulting compound is pharmaceutically acceptable. In certain embodiments, the isotopes of an element are present at a particular position according to their natural abundance. In other embodiments, one or more isotopes of an element at a particular position are enriched beyond their natural abundance.

The term “hydroxy activating group,” as used herein, refers to a labile chemical moiety which is known in the art to activate a hydroxyl group so that it will depart during synthetic procedures such as in a substitution or an elimination reaction. Examples of hydroxyl activating group include, but not limited to, mesylate, tosylate, triflate, p-nitrobenzoate, phosphonate and the like.

The term “activated hydroxyl,” as used herein, refers to a hydroxy group activated with a hydroxyl activating group, as defined above, including, but not limited to mesylate, tosylate, triflate, p-nitrobenzoate, phosphonate groups.

The term “hydroxy protecting group,” as used herein, refers to a labile chemical moiety which is known in the art to protect a hydroxyl group against undesired reactions during synthetic procedures. After said synthetic procedure(s) the hydroxy protecting group as described herein may be selectively removed. Hydroxy protecting groups as known in the art are described generally in P. G. M. Wuts, Greene's Protective Groups in Organic Synthesis, 5th edition, John Wiley & Sons, Hoboken, NJ (2014). Examples of hydroxyl protecting groups include, but are not limited to, benzyloxycarbonyl, 4-methoxybenzyloxycarbonyl, tert-butoxy-carbonyl, isopropoxycarbonyl, diphenylmethoxycarbonyl, 2,2,2-trichloroethoxycarbonyl, allyloxycarbonyl, acetyl, formyl, chloroacetyl, trifluoroacetyl, methoxyacetyl, phenoxyacetyl, benzoyl, methyl, t-butyl, 2,2,2-trichloroethyl, 2-trimethylsilyl ethyl, allyl, benzyl, triphenyl-methyl(trityl), methoxymethyl, methylthiomethyl, benzyloxymethyl, 2-(trimethylsilyl)-ethoxymethyl, methanesulfonyl, trimethylsilyl, triisopropylsilyl, and the like.

The term “protected hydroxy,” as used herein, refers to a hydroxy group protected with a hydroxy protecting group, as defined above, including but not limited to, benzoyl, acetyl, trimethylsilyl, triethylsilyl, methoxymethyl groups, for example.

The term “hydroxy prodrug group,” as used herein, refers to a promoiety group which is known in the art to change the physicochemical, and hence the biological properties of a parent drug in a transient manner by covering or masking the hydroxy group. After said synthetic procedure(s), the hydroxy prodrug group as described herein must be capable of reverting back to hydroxy group in vivo. Hydroxy prodrug groups as known in the art are described generally in Kenneth B. Sloan, Prodrugs, Topical and Ocular Drug Delivery, (Drugs and the Pharmaceutical Sciences; Volume 53), Marcel Dekker, Inc., New York (1992).

The term “amino protecting group,” as used herein, refers to a labile chemical moiety which is known in the art to protect an amino group against undesired reactions during synthetic procedures. After said synthetic procedure(s) the amino protecting group as described herein may be selectively removed. Amino protecting groups as known in the art are described generally in P. G. M. Wuts, Greene's Protective Groups in Organic Synthesis, 5th edition, John Wiley & Sons, Hoboken, NJ (2014). Examples of amino protecting groups include, but are not limited to, methoxycarbonyl, t-butoxycarbonyl, 12-fluorenyl-methoxycarbonyl, benzyloxycarbonyl, and the like.

The term “protected amino,” as used herein, refers to an amino group protected with an amino protecting group as defined above.

The term “leaving group” means a functional group or atom which can be displaced by another functional group or atom in a substitution reaction, such as a nucleophilic substitution reaction. By way of example, representative leaving groups include chloro, bromo and iodo groups; sulfonic ester groups, such as mesylate, tosylate, brosylate, nosylate and the like; and acyloxy groups, such as acetoxy, trifluoroacetoxy and the like.

The term “aprotic solvent,” as used herein, refers to a solvent that is relatively inert to proton activity, i.e., not acting as a proton-donor. Examples include, but are not limited to, hydrocarbons, such as hexane and toluene, for example, halogenated hydrocarbons, such as, for example, methylene chloride, ethylene chloride, chloroform, and the like, heterocyclic compounds, such as, for example, tetrahydrofuran and N-methylpyrrolidinone, and ethers such as diethyl ether, bis-methoxymethyl ether. Such compounds are well known to those skilled in the art, and it will be obvious to those skilled in the art that individual solvents or mixtures thereof may be preferred for specific compounds and reaction conditions, depending upon such factors as the solubility of reagents, reactivity of reagents and preferred temperature ranges, for example. Further discussions of aprotic solvents may be found in organic chemistry textbooks or in specialized monographs, for example: Organic Solvents Physical Properties and Methods of Purification, 4th ed., edited by John A. Riddick et al., Vol. II, in the Techniques of Chemistry Series, John Wiley & Sons, NY, 1986.

The term “protic solvent,” as used herein, refers to a solvent that tends to provide protons, such as an alcohol, for example, methanol, ethanol, propanol, isopropanol, butanol, t-butanol, and the like. Such solvents are well known to those skilled in the art, and it will be obvious to those skilled in the art that individual solvents or mixtures thereof may be preferred for specific compounds and reaction conditions, depending upon such factors as the solubility of reagents, reactivity of reagents and preferred temperature ranges, for example. Further discussions of protogenic solvents may be found in organic chemistry textbooks or in specialized monographs, for example: Organic Solvents Physical Properties and Methods of Purification, 4th ed., edited by John A. Riddick et al., Vol. II, in the Techniques of Chemistry Series, John Wiley & Sons, NY, 1986.

Combinations of substituents and variables envisioned by this invention are only those that result in the formation of stable compounds. The term “stable,” as used herein, refers to compounds which possess stability sufficient to allow manufacture and which maintains the integrity of the compound for a sufficient period of time to be useful for the purposes detailed herein (e.g., therapeutic or prophylactic administration to a subject).

The synthesized compounds can be separated from a reaction mixture and further purified by a method such as column chromatography, high performance liquid chromatography, or recrystallization. As can be appreciated by the skilled artisan, further methods of synthesizing the compounds of the Formula herein will be evident to those of ordinary skill in the art. Additionally, the various synthetic steps may be performed in an alternate sequence or order to give the desired compounds. Synthetic chemistry transformations and protecting group methodologies (protection and deprotection) useful in synthesizing the compounds described herein are known in the art and include, for example, those such as described in R. Larock, Comprehensive Organic Transformations, 2^{nd} Ed. Wiley-VCH (1999); P. G. M. Wuts, Greene's Protective Groups in Organic Synthesis, 5th edition, John Wiley & Sons, Hoboken, NJ (2014); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

The term “subject,” as used herein, refers to an animal. Preferably, the animal is a mammal. More preferably, the mammal is a human. A subject also refers to, for example, a dog, cat, horse, cow, pig, guinea pig, fish, bird and the like.

The compounds of this invention may be modified by appending appropriate functionalities to enhance selective biological properties. Such modifications are known in the art and may include those which increase biological penetration into a given biological system (e.g., blood, lymphatic system, central nervous system), increase oral availability, increase solubility to allow administration by injection, alter metabolism and alter rate of excretion.

The compounds described herein contain one or more asymmetric centers and thus give rise to enantiomers, diastereomers, and other stereoisomeric forms that may be defined, in terms of absolute stereochemistry, as (R)- or(S)-, or as (D)- or (L)- for amino acids. The present invention is meant to include all such possible isomers, as well as their racemic and optically pure forms. Optical isomers may be prepared from their respective optically active precursors by the procedures described above, or by resolving the racemic mixtures. The resolution can be carried out in the presence of a resolving agent, by chromatography or by repeated crystallization or by some combination of these techniques which are known to those skilled in the art. Further details regarding resolutions can be found in Jacques, et al., Enantiomers, Racemates, and Resolutions (John Wiley & Sons, 1981). When the compounds described herein contain olefinic double bonds, other unsaturation, or other centers of geometric asymmetry, and unless specified otherwise, it is intended that the compounds include both E and Z geometric isomers or cis- and trans-isomers. Likewise, all tautomeric forms are also intended to be included. Tautomers may be in cyclic or acyclic. The configuration of any carbon-carbon double bond appearing herein is selected for convenience only and is not intended to designate a particular configuration unless the text so states; thus a carbon-carbon double bond or carbon-heteroatom double bond depicted arbitrarily herein as trans may be cis, trans, or a mixture of the two in any proportion.

Certain compounds of the present invention may also exist in different stable conformational forms which may be separable. Torsional asymmetry due to restricted rotation about an asymmetric single bond, for example because of steric hindrance or ring strain, may permit separation of different conformers. The present invention includes each conformational isomer of these compounds and mixtures thereof.

As used herein, the term “pharmaceutically acceptable salt,” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66:2-19 (1977). The salts can be prepared in situ during the final isolation and purification of the compounds of the invention, or separately by reacting the free base function with a suitable organic acid. Examples of pharmaceutically acceptable salts include, but are not limited to, nontoxic acid addition salts are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. Other pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentane-propionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate.

As used herein, the term “pharmaceutically acceptable ester” refers to esters which hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include, but are not limited to, formates, acetates, propionates, butyrates, acrylates and ethylsuccinates.

### Abbreviations

Abbreviations which may be used in the descriptions of the scheme and the examples that follow are:
ACN for acetonitrile;
CDI for 1,1′-carbonyldiimidazole;
DABCO for 1,4-diazabicyclo[2.2.2]octane;
DBU for 1,8-diazabicyclo[5.4.0]undec-7-ene;
DMAP for dimethylaminopyridine;
DMF for dimethyl formamide;
DMSO for dimethyl sulfoxide;
DCM for dichloromethane;
NMP for N-Methyl-2-pyrrolidone;
AcOH for acetic acid;
EtOAc or EA for ethyl acetate;
HCl for hydrogen chloride;
HBr for hydrogen bromide;
NaHCO₃ for sodium bicarbonate;
HPLC for high-pressure liquid chromatography;
MeOH for methanol;
MTBE for tert-butyl methyl ether;
EtOH for ethanol;
NMM for N-methylmorpholine;
HATU for 1-[bis(dimethylamino)methylene]-1H-1,2,3-triazolo[4,5-b]pyridinium 3-oxide hexafluorophosphate;
TEA for triethylamine;
THF for tetrahydrofuran;
2-MeTHF for 2-methyltetrahydrofuran;
TFAA for trifluoroacetic anhydride;
TsOH for p-toluenesulfonic acid;
TsCl for p-toluenesulfonyl chloride;
TPP or PPh₃ for triphenylphosphine;
T₃P for n-propanephosphonic acid anhydride;
Boc for t-butoxycarbonyl;
Bn for benzyl;
Ph for phenyl;
PhMe for toluene.

All other abbreviations used herein, which are not specifically delineated above, shall be accorded the meaning which one of ordinary skill in the art would attach.

### Synthetic Scheme

The present invention will be better understood with Schemes 1. It will be readily apparent to one of ordinary skill in the art that the process of the present invention can be practiced by substitution of the appropriate reactants and that the order of the steps themselves can be varied.

A synthetic route to Compound (I) is summarized in Scheme 1. Compound A can react with 1,1′-carbonyldiimidazole to produce compound (B). Compound (B) then couples with compound (W) to produce compound (C). Compound (C) is cyclized with a dehydration agent to produce Compound (I), which is isolated via crystallization to provide a crystalline product. The product is recrystallized from acetone and ethanol to provide Compound (I).

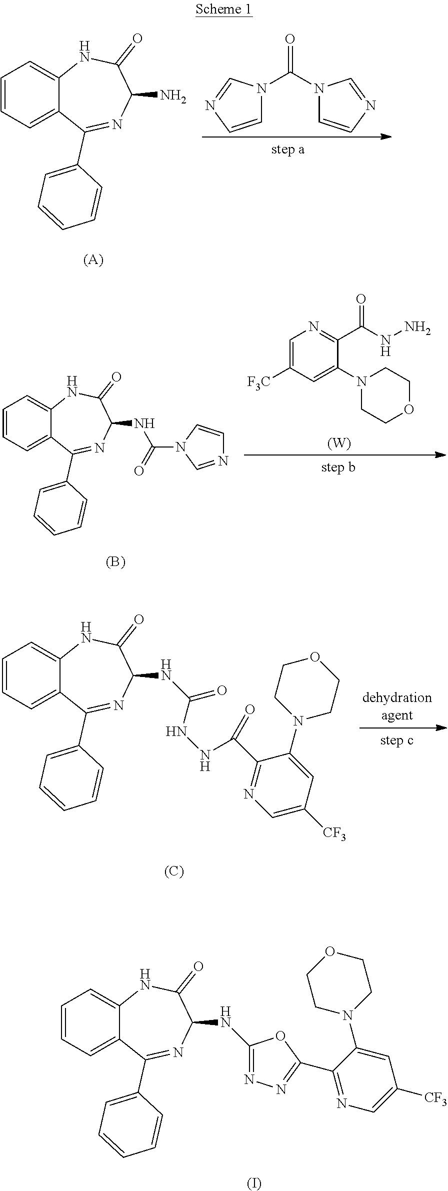

The compounds and processes of the present invention will be understood in connection with the following illustrative methods by which the compounds of the invention may be prepared. It will be understood that any of the reactions described herein, in any of its variations, can be combined with one or more of the other reactions, in any of their respective variations, substantially in analogy with Scheme 1 above.

### Examples

The compounds and processes of the present invention will be better understood in connection with the following examples, which are intended as an illustration only and not limiting of the scope of the invention. Various changes and modifications to the disclosed embodiments will be apparent to those skilled in the art and such changes and modifications including, without limitation, those relating to the chemical structures, substituents, derivatives, formulations and/or methods of the invention may be made without departing from the spirit of the invention and the scope of the appended claims.

### Example 1. Preparation of(S)—N-(2-oxo-5-phenyl-2,3-dihydro-1H-benzo[e][1,4]diazepin-3-yl)-1H-imidazole-1-carboxamide (B)

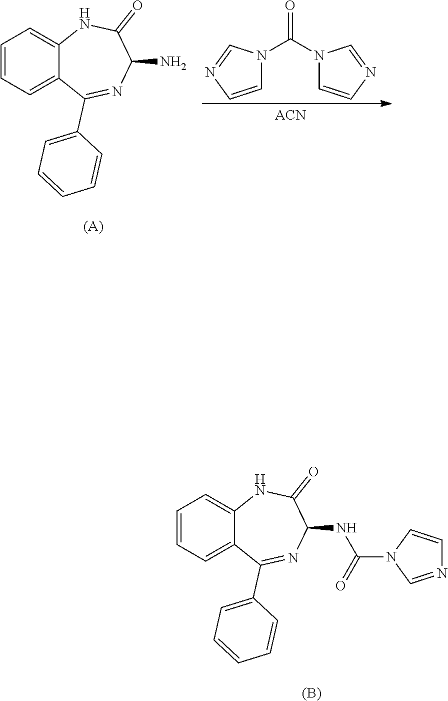

A reactor equipped with an overhead stirrer was charged with CDI (16.6 kg, 102 mol, 1.51 equiv) and ACN (153 L). The mixture was stirred at 40° C. for 1 hour, resulting in the formation of a clear solution. A suspension of compound A (17 kg, 68 mol) in ACN (102 L) was then added to the reaction at 40° C. over 1.5 hours. Once the addition was complete, the reaction was allowed to stir at 40° C. for 4 hours. The reaction progress was monitored by HPLC. Upon completion, the reaction batch was cooled to 0° C., and a mixture of ACN and water (10:1, 93.5 L) was slowly added over 1 hour. The resulting suspension was stirred at 0° C. for 0.5 hour and was then warmed to room temperature over 2 hours. Subsequently, the white solid was filtered off under vacuum, rinsed with ACN (2×34 L). The wet-cake was transferred to a vacuum oven and dried in-vacuo at 42° C. overnight to afford(S)—N-(2-oxo-5-phenyl-2,3-dihydro-1H-benzo[e][1,4]diazepin-3-yl)-1H-imidazole-1-carboxamide (22.9 kg, 66.3 mol, 97.5% yield).

¹H NMR (400 MHZ, DMSO-d₆) δ 11.03 (s, 1H), 9.83 (d, J=7.5 Hz, 1H), 8.47 (s, 1H), 7.94 (s, 1H), 7.67 (ddd, J=8.5, 7.1, 1.7 Hz, 1H), 7.58-7.51 (m, 3H), 7.50-7.43 (m, 2H), 7.35 (ddd, J=11.7, 8.1, 1.4 Hz, 2H), 7.30-7.24 (m, 1H), 7.08 (s, 1H), 5.36 (d, J=7.5 Hz, 1H).

LC-MS(ESI, m/z): 346.13 [M+H]⁺.

### Example 2. Preparation of(S)-2-(3-morpholino-5-(trifluoromethyl) picolinoyl)-N-(2-oxo-5-phenyl-2,3-dihydro-1H-benzo[e][1,4]diazepin-3-yl) hydrazine-1-carboxamide (C)

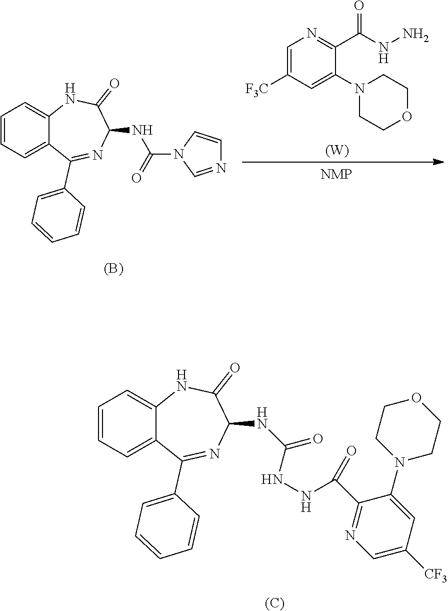 [SMILES: O=C1Nc2ccccc2C(c2ccccc2)=N[C@@H]1NC(=O)n1ccnc1 ; NNC(=O)c1ncc(C(F)(F)F)cc1N1CCOCC1 ; "(W)" ; "(B)" ; O=C(NNC(=O)c1ncc(C(F)(F)F)cc1N1CCOCC1)N[C@H]1N=C(c2ccccc2)c2ccccc2NC1=O ; "(C)"]

A reactor equipped with an overhead stirrer was charged with compound W (19.5 kg, 67.2 mol, 1.05 equiv) and NMP (66 L). The mixture was stirred at 35° C. for 1 hour, resulting in the formation of a clear solution. Compound B (21.9 kg, 63.4 mol) was added to the reaction as solid in 4 portions over 2 hours. The reaction was then stirred at 35° C. for 30 hours. The reaction progress was monitored by HPLC. Upon completion, the crude reaction was diluted with EtOAc (197 L) and then washed with 5% brine solution (438 L). The aqueous layer was extracted with EtOAc (110 L), and the combined organic layer was washed with 5% brine (3×175 L). Next, the organic solution was concentrated to approximately 110 L under vacuum at 40° C., followed by the addition of 110 L EtOH. This concentration and addition process was repeated no less than 3 times until the solvent was swapped to EtOH. n-Heptane (548 L) was then slowly added to the EtOH solution at 20° C. over 1 hour. The resulting suspension was stirred at 20° C. for 3 hours. Subsequently, the yellow solid was filtered off under vacuum, rinsed with n-heptane (44 L). The wet-cake was transferred to a vacuum oven and dried in-vacuo at 42° C. overnight to afford(S)-2-(3-morpholino-5-(trifluoromethyl) picolinoyl)-N-(2-oxo-5-phenyl-2,3-dihydro-1H-benzo[e][1,4]diazepin-3-yl) hydrazine-1-carboxamide (31.5 kg, 55.5 mol, 87.5% yield). 1H NMR (400 MHZ, DMSO-d₆) δ 10.96 (s, 1H), 10.32 (br-s, 1H), 8.61 (br-s, 1H), 8.48 (s, 1H), 7.73 (s, 1H), 7.66 (ddd, J=8.6, 7.1, 1.7 Hz, 1H), 7.56-7.37 (m, 6H), 7.35-7.31 (m, 2H), 7.30-7.23 (m, 1H), 5.14 (d, J=8.3 Hz, 1H), 3.66 (dd, J=6.0, 3.5 Hz, 4H), 3.17 (dd, J=6.0, 3.5 Hz, 4H).

LC-MS(ESI, m/z): 568.19 [M+H]⁺.

### Example 3. Preparation of(S)-3-((5-(3-morpholino-5-(trifluoromethyl) pyridin-2-yl)-1,3,4-oxadiazol-2-yl)amino)-5-phenyl-1,3-dihydro-2H-benzo[e][1,4]diazepin-2-one (I)

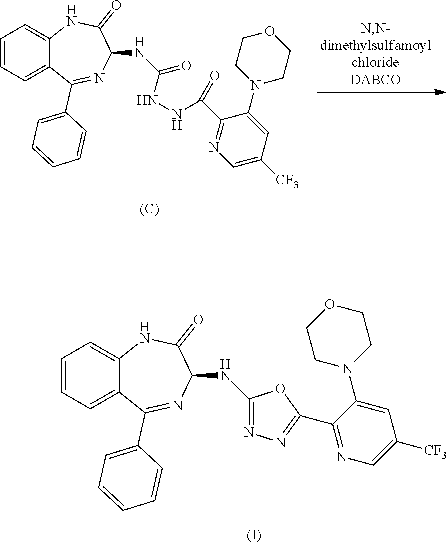 [SMILES: O=C(NNC(=O)c1ncc(C(F)(F)F)cc1N1CCOCC1)N[C@H]1N=C(c2ccccc2)c2ccccc2NC1=O ; "(C)" ; O=C1Nc2ccccc2C(c2ccccc2)=N[C@@H]1Nc1nnc(-c2ncc(C(F)(F)F)cc2N2CCOCC2)o1 ; "(I)"]

A reactor equipped with an overhead stirrer was charged with compound C (26.0 kg, 45.8 mol) and THF (260 L). The mixture was stirred at 25° C. for 1 hour, resulting in the formation of a clear solution. DABCO (20.0 kg, 178 mol, 4 equiv) was added to the solution. Next, dimethylsulfamoyl chloride (13.9 kg, 96.8 mol, 2.1 equiv) was slowly added over 4 hours at 25° C. Once the addition was complete, the mixture was allowed to stir for 15 hours. The reaction progress was monitored by HPLC. Upon completion, the crude reaction was diluted with EtOAc (260 L) and quenched with the addition of 0.2 N HCl (208 L). The organic layer was then washed with 5% NaHCO₃ solution (260 L) and 10% brine (250 L). Next, the organic layer was concentrated under vacuum to afford the crude mixture. The crude mixture was dissolved in EtOAc (104 L) at 35° C., and stirred for 16 hours at 25° C., resulting in the formation of a slurry. Subsequently, MTBE (104 L) was gradually added to the slurry over 2 hours. Once the addition was complete, the mixture was cooled to 0° C. and stirred at this temperature for 5 hours. The crude solid was collected through filtration in vacuum and was washed with cold MTBE/EtOAc mixture (1:1 52 L).

The crude solid was dissolved in acetone (612 L) at 25° C., resulting in the formation of a homogeneous solution. The solution was concentrated to approximately 102 L under vacuum at 30° C. 0.5% wt seed (compound I, 102 g) was charged to the solution and the mixture was stirred at 25° C. for 1 hour, resulting in the formation of a suspension. EtOH (102 L) was then added, and the suspension was concentrated to approximately 102 L under vacuum at 30° C. This concentration and addition process was repeated at least 5 times until the acetone was completely swapped to EtOH. Next, the batch temperature was cooled to 25° C. and the suspension was stirred at this temperature for 24 hours. The yellow solid was collected by filtration and washed with EtOH (26 L). The wet-cake was transferred to a vacuum oven and dried in-vacuo at 42° C. overnight to afford(S)-3-((5-(3-morpholino-5-(trifluoromethyl) pyridin-2-yl)-1,3,4-oxadiazol-2-yl)amino)-5-phenyl-1,3-dihydro-2H-benzo[e][1,4]diazepin-2-one (18.1 kg, 33.0 mol, 99.8% purity, 72.1% yield). ¹HNMR (400 MHz, DMSO-d₆): δ 10.98 (br-s, 1H), 9.40 (d, J=8.0 Hz, 1H), 8.69 (br-d, J=4.0 Hz, 1H), 7.89 (d, J=4.0 Hz, 1H), 7.68 (dt, J=8.0, 4.0 Hz, 1H), 7.56-7.51 (m, 3H), 7.49-7.45 (m, 2H), 7.38-7.35 (m, 2H), 7.29 (br-t, J=8.0 Hz, 1H) 5.22 (d, J=8.0 Hz, 1H), 3.75-3.72 (m, 4H), 3.09-3.07 (m, 4H);

LC-MS(ESI, m/z): 550.17 [M+H]⁺.

While this invention has been particularly shown and described with references to preferred embodiments thereof, it will be understood by those skilled in the art that various changes in form and details may be made therein without departing from the scope of the invention encompassed by the appended claims.

1. A process for producing Compound (I), 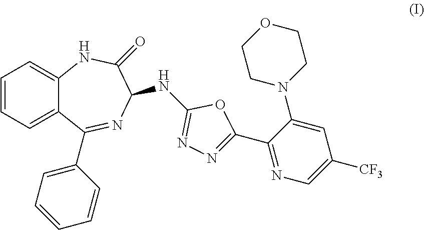 [SMILES: O=C1Nc2ccccc2C(c2ccccc2)=N[C@@H]1Nc1nnc(-c2ncc(C(F)(F)F)cc2N2CCOCC2)o1]
said process comprising the steps of
(a) reacting compound (A) with 1,1′-carbonyldiimidazole, to produce compound (B): 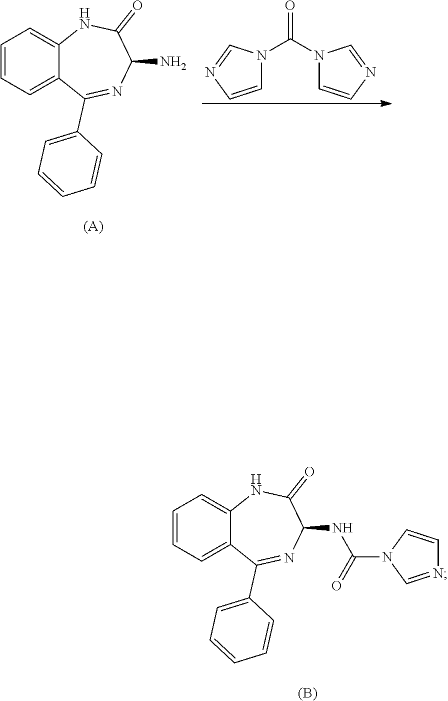
(b) reacting compound (B) with compound (W), to produce compound (C): 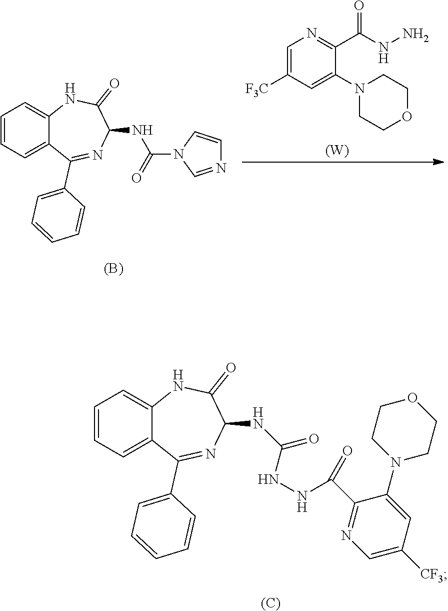 [SMILES: O=C1Nc2ccccc2C(c2ccccc2)=N[C@@H]1NC(=O)n1ccnc1 ; NNC(=O)c1ncc(C(F)(F)F)cc1N1CCOCC1 ; "(W)" ; "(B)" ; O=C(NNC(=O)c1ncc(C(F)(F)F)cc1N1CCOCC1)N[C@H]1N=C(c2ccccc2)c2ccccc2NC1=O ; "(C)"]
and
(c) reacting compound (C) with a dehydration agent selected from the group consisting of N,N-dimethylsulfamoyl chloride, methyl N-(triethylammoniumsulfonyl) carbamate, phosphorus trichloride, and phosphorus pentachloride, to produce Compound (I):

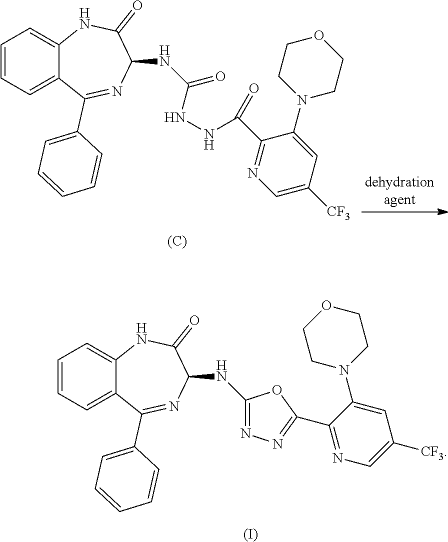 [SMILES: O=C(NNC(=O)c1ncc(C(F)(F)F)cc1N1CCOCC1)N[C@H]1N=C(c2ccccc2)c2ccccc2NC1=O ; "(C)" ; O=C1Nc2ccccc2C(c2ccccc2)=N[C@@H]1Nc1nnc(-c2ncc(C(F)(F)F)cc2N2CCOCC2)o1 ; "(I)"]

2. The process of claim 1, wherein step (c) is conducted in the presence of a base.

3. The process of claim 2, wherein the base is selected from the group consisting of 1,4-diazabicyclo[2.2.2]octane, triethylamine, 1,8-diazabicyclo[5.4.0]undec-7-ene, 1-methylimidazole, diisopropylethylamine, sodium bicarbonate, potassium phosphate, sodium hydroxide and N-methylmorpholine.

4. The process of claim 1, wherein the dehydration agent is N,N-dimethylsulfamoyl chloride.

5. The process of claim 1, wherein the dehydration agent is N,N-dimethylsulfamoyl chloride and step (c) is conducted in the presence of 1,8-diazabicyclo[5.4.0]undec-7-ene.

6. The process of claim 1, wherein step (a) is conducted in a solvent selected from the group consisting of acetonitrile, dichloromethane, dimethylformamide, dimethyl sulfoxide, tetrahydrofuran, dimethylacetamide, N-methyl-2-pyrrolidone, sulfolane, dioxane and mixtures of two or more thereof.

7. The process of claim 6, wherein step (a) is conducted in acetonitrile.

8. The process of claim 6, wherein step (a) is conducted at a temperature of about 20° C. to about 80° C.

9. The process of claim 8, wherein step (a) is conducted at a temperature of about 40° C.

10. The process of claim 1, wherein step (b) is conducted in a solvent selected from the group consisting of acetonitrile, dimethylformamide, dimethylsulfoxide, tetrahydrofuran, N-methyl-2-pyrrolidone, sulfolane, dioxane and mixtures of two or more thereof.

11. The process of claim 10, wherein step (b) is conducted in N-methyl-2-pyrrolidone.

12. The process of claim 10, wherein step (b) is conducted at a temperature of about 10° C. to about 50° C., about 30° C. to about 50° C., about 30° C. to about 40° C. or about 35° C.

13. The process of claim 1, further comprising recrystallizing Compound (I).

14. The process of claim 13, wherein Compound (I) is recrystallized by
(i) Dissolving Compound (I) in a first solvent selected from the group consisting of ethyl acetate, ethanol, methanol, 2-methyltetrahydrofuran, dimethyl sulfoxide, dimethylformamide, and mixtures of two or more thereof; and
(ii) Replacing the solvent of step (i) with a second solvent selected from the group consisting of toluene, heptane, ethanol, isopropanol, methyl t-butyl ether, water and mixtures of two or more thereof, thereby inducing crystallization of Compound (I);
provided that when the first solvent is ethanol, the second solvent is not ethanol.

15. The process of claim 14, wherein the first solvent is acetone, ethanol or a combination thereof.

16. The process of claim 14, wherein the second solvent is ethanol, heptane or methyl t-butyl ether.
